(19) United States
(12) Patent Application Publication
(10) Pub. No.: US 2025/0049493 A1
(43) Pub. Date: Feb. 13, 2025

(54) PROTECTIVE WORKING CHANNELS FOR ABLATION PROCEDURES AND RELATED METHODS

(71) Applicant: CUSTOM MEDICAL APPLICATIONS INC., Farmers Branch, TX (US)

(72) Inventors: Ricardo Ruiz-Lopez, Barcelona (ES); Nicholas Sandor Racz, Farmers Branch, TX (US)

(21) Appl. No.: 18/799,410

(22) Filed: Aug. 9, 2024

Related U.S. Application Data
(60) Provisional application No. 63/518,509, filed on Aug. 9, 2023.

(51) Int. Cl. A61B 18/14; A61B 18/00

(52) U.S. Cl. CPC A61B 18/14; A61B 2018/00077; A61B 2018/00083; A61B 2018/00095; A61B 2018/00184; A61B 2018/00577; A61B 2018/144

(57) ABSTRACT

Protective working channels useful in ablative procedures may include a cannula having a shield at a distal end of the cannula. The shield may be sized, shaped, and positioned to cover a first portion of an ablative tip of an ablation probe introducible through the cannula and to expose a second, different portion of the ablative tip of the ablation probe. An electrically insulating material may be disposed on at least one surface of the cannula at least at the distal end of the cannula, including the shield. When using the protective working channels, an ablative tip of an ablation probe may be positioned proximate to a target tissue within the subject, with the ablative tip partially exposed at a distal end of the cannula and the ablative tip interposed between the target tissue and a shield at the distal end of the cannula.

### CROSS-REFERENCE TO RELATED APPLICATION

This application claims the benefit under 35 U.S.C. § 119 (e) of U.S. Provisional Patent Application Ser. No. 63/518,509, filed Aug. 9, 2023, the disclosure of which is hereby incorporated herein in its entirety by this reference.

### FIELD

This disclosure relates generally to protective working channels for ablation instruments and related methods. More specifically, disclosed embodiments relate to protective working channels which may better facilitate precise ablation of target tissues, reduce damage to healthy tissues proximate to the target tissues, and enable aggressive ablation of target tissues while mitigating risks to surrounding healthy tissues.

### BACKGROUND

Surgical procedures involving ablation of a target tissue are generally extremely disruptive and may cause a great deal of damage to healthy tissue. In some instances, the target volume is small, while the minimum volume ablatable by an ablative tip, end, or edge of an ablation probe may be larger, resulting in collateral damage to nearby healthy tissue. In an effort to mitigate damage to nearby healthy tissue, directionally targetable ablative tips, ends, or edges may be utilized for the ablation probe, as disclosed in U.S. Pat. No. 9,265,563, issued Feb. 23, 2016, and listing the same inventors as those listed on this application, the contents of which are incorporated herein in their entirety by this reference.

### BRIEF SUMMARY

In some examples, protective working channels useful in ablative procedures include a cannula having a shield at a distal end of the cannula. The shield may be sized, shaped, and positioned to cover a first portion of an ablative tip of an ablation probe introducible through the cannula and to expose a second, different portion of the ablative tip of the ablation probe. An electrically insulating material may be disposed on at least one surface of the cannula at least at the distal end of the cannula, including the shield.

In other examples, kits for performing ablation procedures may include a protective working channel having a cannula including a shield at a distal end of the cannula. The protective working channel may also include an electrically insulating material disposed on at least one surface of the cannula at least at the distal end of the cannula, including the shield. The kit may further include an introducer needle introducible through the cannula and an ablation probe introducible through the cannula, through the introducer needle, or through the cannula and through the introducer needle. The shield may be sized, shaped, and positioned to cover a first portion of an ablative tip of the ablation probe and to expose a second, different portion of the ablative tip of the ablation probe when the ablation probe is at least partially inserted into the cannula, the ablative tip is located proximate to the distal end of the cannula, and the ablative tip is oriented for performing an ablation procedure.

In other examples, methods of making protective working channels may involve positioning a shield at a distal end of a cannula. The shield may be sized, shaped, and positioned to cover a first portion of an ablative tip of an ablation probe introducible through the cannula and to expose a second, different portion of the ablative tip of the ablation probe. An electrically insulating material may be disposed on at least one surface of the cannula at least at the distal end of the cannula, including the shield.

In still other examples, methods of using protective working channels may involve inserting a protective working channel comprising a cannula into a subject. An ablation probe may be introduced into the subject utilizing the cannula. An ablative tip of the ablation probe may be placed proximate to a target tissue within the subject, with the ablative tip partially exposed at a distal end of the cannula and the ablative tip interposed between the target tissue and a shield at the distal end of the cannula. An electrically insulating material may be disposed on at least one surface of the cannula at least at the distal end of the cannula, including the shield.

### BRIEF DESCRIPTION OF THE DRAWINGS

While this disclosure concludes with claims particularly pointing out and distinctly claiming specific embodiments, various features and advantages of embodiments within the scope of this disclosure may be more readily ascertained from the following description when read in conjunction with the accompanying drawings. In the drawings:

FIG. 1 is a side perspective view of a protective working channel;

FIG. 2 is a side perspective view of an assembly including the protective working channel of FIG. 1;

FIG. 3 is an enlarged side perspective view of an ablative tip of the assembly of FIG. 2;

FIG. 4 is a flowchart showing a method of making a protective working channel; and

FIG. 5 is another flowchart showing a method of using a protective working channel.

### DETAILED DESCRIPTION

The illustrations presented in this disclosure are not meant to be actual views of any particular protective working channel, kit, assembly or component thereof, but are merely idealized representations employed to describe illustrative embodiments. Thus, the drawings are not necessarily to scale.

Disclosed embodiments relate generally to protective working channels which may better facilitate precise ablation of target tissues, may reduce damage to healthy tissues proximate to the target tissues, may enable aggressive ablation of target tissues while mitigating risks to surrounding healthy tissues. More specifically, disclosed are embodiments of protective working channels which may, for example, include shields positioned and configured to partially expose an ablative tip to a target tissue and to partially shield other tissue from the ablative tip. An electrically insulating material may be disposed on at least a portion of the shield to inhibit the flow of heat from the ablative tissue toward the other tissue and to protect the other tissue from the high temperatures associated with ablating the target tissue.

As used herein, the terms “substantially” and “about” in reference to a given parameter, property, or condition means and includes to a degree that one of ordinary skill in the art would understand that the given parameter, property, or condition is met with a degree of variance, such as within acceptable manufacturing tolerances. For example, a parameter that is substantially or about a specified value may be at least about 90% the specified value, at least about 95% the specified value, at least about 99% the specified value, or even at least about 99.9% the specified value.

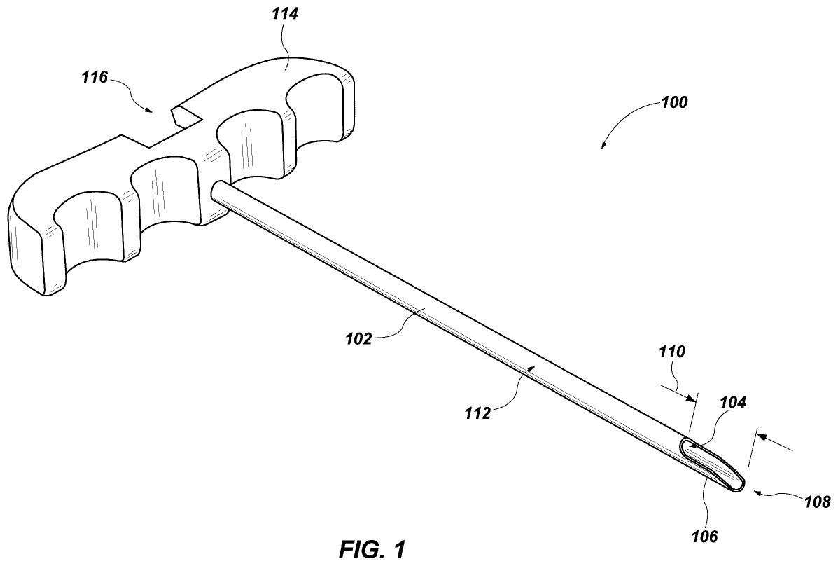

FIG. 1 is a side perspective view of a protective working channel 100. The protective working channel 100 may, for example, be used in an ablation procedure. For example, the protective working channel 100 may be used for ablation procedures conducted on the nervous system and adjacent tissues, such as for pain management purposes. More specifically, the protective working channel 100 may be used for ablation procedures performed on the meningeal sheath (sometimes also referred to as a “sleeve”), the peripheral nervous system, and the spinal canal. As another example, the protective working channel 100 may be used for ablation procedures conducted on connective tissue, the skin, the upper digestive system, the muscles, and the joints. More broadly, the protective working channel 100 may be used for ablation procedures performed on any tissue within the endodermal, ectodermal, or mesodermal sheaths of a subject, which initially form during embryonic development.

The protective working channel 100 may include a cannula 102. For example, the cannula 102 may include a tube or other hollow member, sized and shaped for partial introduction into a subject and to enable other instruments to be introduced into the subject through the cannula 102. More specifically, the cannula 102 may have a circular, elliptical, or oval cross-sectional shape, when viewed in a cross-section taken at least substantially perpendicular to a direction of insertion of a leading portion of the cannula 102 into a subject, and may define an interior space 104 through which other instruments may be inserted.

The cannula 102 may include a biocompatible material, such as, for example, those suitable for medical devices and for exposure to the tissues of a subject during an ablation procedure. More specifically, the cannula 102 may include or consist of one or more materials selected from the group consisting of stainless steel, cobalt-chromium alloy, polyvinylchloride, polyethylene, polypropylene, polytetrafluoroethylene, polymethylmethacrylate, titanium, a titanium alloy, alumina, zirconia, and/or graphene in a binder material.

In some examples, the cannula 102 may be rigid. For example, the cannula 102 may include or be formed from a metal or ceramic material. More specifically, the cannula 102 may, for example, resist substantial deformation during an ablation procedure and may be intended for insertion into a subject oriented directly toward a subject tissue (e.g., straight insertion in examples where the cannula 102 is straight, curved insertion in examples where the cannula 102 is curved). In other examples, the cannula 102 may be flexible. For example, the cannula 102 may include or be formed from a polymer material. More specifically, the cannula 102 may, for example, substantially deform during or prior to an ablation procedure to facilitate insertion.

The cannula 102 may include a shield 106 located at or proximate to a distal end 108 of the cannula 102. The shield 106 may be, for example, sized, shaped, and positioned to cover a first portion of an ablative tip of an ablation probe introducible through the cannula and to expose a second, different portion of the ablative tip of the ablation probe. More specifically, a majority of the cannula 102 may define an enclosed space 104 and the shield 106 may only partially enclose or surround a volume at the distal end 108 of the cannula 102. As a specific, nonlimiting example, the shield 106 may include an extension or protrusion from a remainder of the cannula 102, extending angularly only partially around a perimeter of the remainder of the cannula 102 to define a scoop or spoon shape.

The shield 106 may be sized, shaped, and positioned to cover, for example, about one-third or more of an exterior of an ablative tip of an ablation probe, and to expose a remainder of the ablative tip, when the ablative tip is located proximate to the shield 106 and positioned for an ablation procedure. More specifically, the shield 106 may be sized, shaped, and positioned to cover, for example, about half or more of an exterior of an ablative tip of an ablation probe, and to expose a remainder of the ablative tip, when the ablative tip is located proximate to the shield 106 and positioned for an ablation procedure. As specific, nonlimiting examples, at least a portion of the shield 106 may extend around about 120 degrees, about 150 degrees, about 180 degrees, about 210 degrees, 240 degrees, or about 270 degrees of an exterior of an ablative tip of an ablation probe when the ablative tip is located proximate to the shield 106 and positioned for an ablation procedure, as viewed in a cross-sectional view at least substantially perpendicular to a direction of insertion of the distal end 108.

A length 110 of the shield 106, as measured in a direction at least substantially parallel to a direction of insertion of the distal end 108, may be sufficient to ensure that an ablative tip of an ablation probe is exposed to a target tissue without having to extend the ablative tip beyond the distal end 108 of the cannula 102. For example, the length 110 of the shield 106 may be, for example, equal to or greater than a corresponding length of the ablative tip of an ablation probe, or of a lesion wire of the ablation tip, to be used with the protective working channel 100. More specifically, the length 110 of the shield 106 may be, for example, between about 5% and about 15% (e.g., about 7%, about 10%, about 12%) of a length of the cannula 102, as measured in the same direction. As specific, nonlimiting examples, the length 110 of the shield 106 may be between about 0.3 inch (7.6 mm) and about 0.7 inch (17.8 mm) (e.g., about 0.4 inch (10.2 mm), about 0.5 inch (12.7 mm), about 0.6 inch (15.2 mm)).

An electrically insulating material 112 may be disposed on at least one surface of the cannula 102 to inhibit conduction of energy during an ablation procedure from flowing to tissues other than the target tissue. For example, the electrically insulating material 112 may be disposed on one or more surfaces of the cannula 102 at and proximate to the distal end 108, including on at least one surface defining the shield 106. In some examples, the electrically insulating material 112 may be disposed on at least substantially all surfaces of the cannula 102. In other examples, the electrically insulating material 112 may be disposed on at least substantially all external surfaces of the cannula 102, at least substantially all internal surfaces of the cannula 102, or at least substantially all external and at least substantially all internal surfaces of the cannula 102.

The electrically insulating material 112 may include, for example, one or more ceramic, polymer, or composite materials. More specifically, the electrically insulating material 112 may include, for example, a porcelain material or a polyester material. In examples where the ablation probe is configured to utilize radio-frequency (RF) alternating current when conducting an ablation procedure, the electrically insulating material 112 may be positioned and configured to impede flow of RF current from the ablation probe to tissues other than the target tissue. In some examples, the electrically insulating material 112 may also exhibit thermally insulating properties.

The cannula 102 of the protective working channel 100 may be sized and shaped to enable an introducer needle and an ablation probe to pass at least partially through the cannula 102, either sequentially or concurrently. For example, an inner diameter of the cannula 102 may be sized to receive a 16 gauge or larger probe within the cannula 102. More specifically, the inner diameter of the cannula 102 may be, for example, between about 0.05 inch (1.3 mm) and about 0.2 inch (5.1 mm) (e.g., about 0.06 inch (1.5 mm), about 0.07 inch (1.8 mm), about 0.1 inch (2.5 mm)).

In some examples, the cannula 102 may include a handle 114 at a proximal end 116 of the cannula 102 to enable a user to more easily hold, introduce, position, orient, and otherwise manipulate the protective working channel 100, including during an ablation procedure. In examples where the cannula 102 includes a handle 114, the electrically insulating material 112 may not be disposed on at least exterior surfaces of the cannula 102 at the proximal end 116 to enable a material of the handle 114 to better remain affixed to the material of the cannula 102.

A length of the cannula 102, as measured from a point of contact with the handle 114 to the distal end 108 in a direction at least substantially parallel to a direction of insertion of the cannula 102, may be sufficient to enable the cannula 102 to reach a target tissue within a subject, such as, for example, tissues within the regions discussed previously herein. More specifically, the length of the cannula 102 may be, for example, between about 2 inches (5.1 cm) and about 14 inches (35.6 cm). As a specific, nonlimiting example, the length of the cannula 102 may be between about 4 inches (10.2 cm) and about 12 inches (30.5 cm) (e.g., about 6 inches (15.2 cm), about 8 inches (20.3 cm), about 10 inches (25.4 cm)).

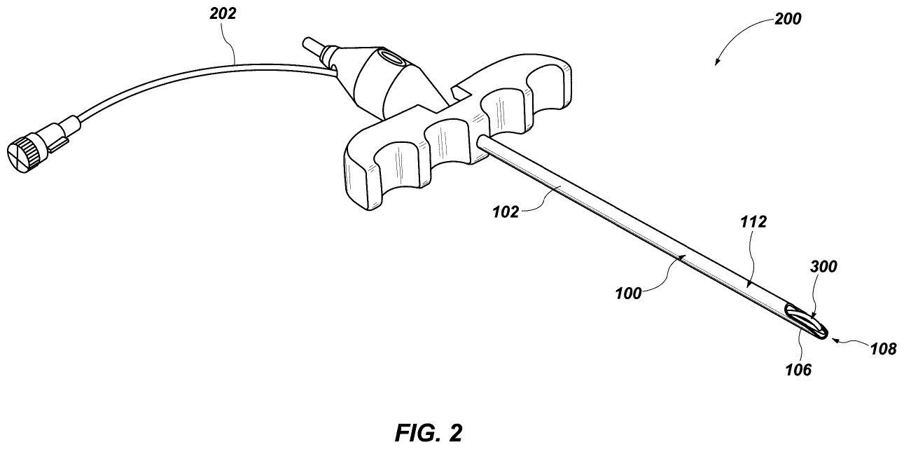

FIG. 2 is a side perspective view of an assembly 200 including the protective working channel 100 of FIG. 1. As shown and discussed previously, the protective working channel 100 may include a cannula 102 having a shield 106 at a distal end 108 of the cannula 102. An electrically insulating material 112 may be disposed on at least one surface of the cannula 102 at least at the distal end 108 of the cannula 102, including the shield 106.

The assembly 200 may be put together from a kit including the protective working channel 100. For example, the kit may include an introducer needle introducible from the cannula 102 of the protective working channel 100. The introducer needle may be longer than the cannula 102, so that a piercing tip of the introducer needle may extend beyond the distal end 108 of the cannula 102. The piercing tip may be utilized to introduce the assembly 200 into a subject, and advancement of the assembly 200 may cause the cannula 102 to enter the subject, starting with the distal end 108. After introduction into the subject, and optionally after the distal end 108 of the cannula 102 is positioned proximate to a target tissue within the subject, the introducer needle may be withdrawn from the cannula 102.

The kit, and the resulting assembly 200, may include an ablation probe 202 for performing an ablation procedure. The ablation probe 202 may likewise be introducible through the cannula 102, and optionally through the introducer needle before withdrawal of the introducer needle. Stated another way, the ablation probe 202 may be introducible through the cannula 102, through the introducer needle, or through the cannula 102 and through the introducer needle.

The shield 106 may be sized, shaped, and positioned to cover a first portion of an ablative tip 300 of the ablation probe and to expose a second, different portion of the ablative tip 300 of the ablation probe 202 when the ablation probe 202 is at least partially inserted into the cannula 102, the ablative tip 300 is located proximate to the distal end 108 of the cannula 102, and the ablative tip 300 is oriented for performing an ablation procedure.

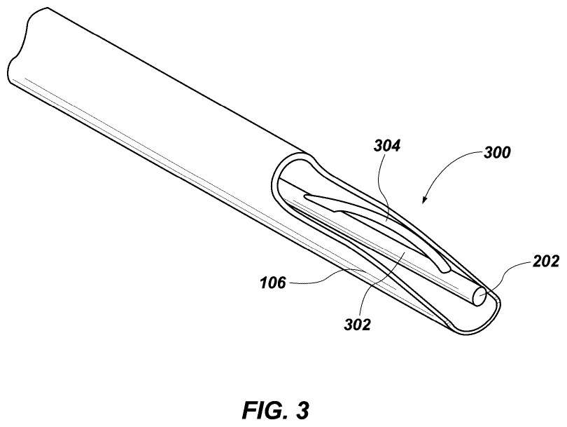

FIG. 3 is an enlarged side perspective view of an ablative tip 300 of the assembly 200 of FIG. 2. The ablation probe 202 may include a body 302 and a lesion wire 304. For a majority of the length of the ablation probe 202, the lesion wire 304 may be located within the body 302. At the ablative tip 300, the lesion wire 304 may extend through a port in the body 302, along one peripheral side of the body 302 at the exterior of the body 302, and back through another port into the body 302. In some examples, the ablation probe 202 may be at least substantially consistent with examples of ablation probes disclosed in U.S. Pat. No. 9,265,563, issued Feb. 23, 2016, and listing the same inventors as those listed on this application

By exposing the lesion wire 304 solely at a selected circumferential location around the ablative tip 300, energy from the lesion wire 304 for ablating a target tissue may be directionally focused toward the target tissue. For example, the lesion wire 304 of the ablative tip 300 may be exposed at a first angular position around a circumference of the ablative tip 300, and a second angular position of the ablative tip, offset from the first angular position by 180 degrees, may lack any exposed lesion wire.

When the ablative tip 300 is located proximate to the shield 106 and oriented for an ablation procedure, the body 302 of the ablation probe 202 may be interposed between the lesion wire 304 and the shield 106. Stated another way, the lesion wire 304 may face toward a target tissue, and the ablative tip 300 may be interposed between the target tissue and the shield 106 at the distal end 108 of the cannula 102, when the ablative tip 300 is oriented for an ablation procedure.

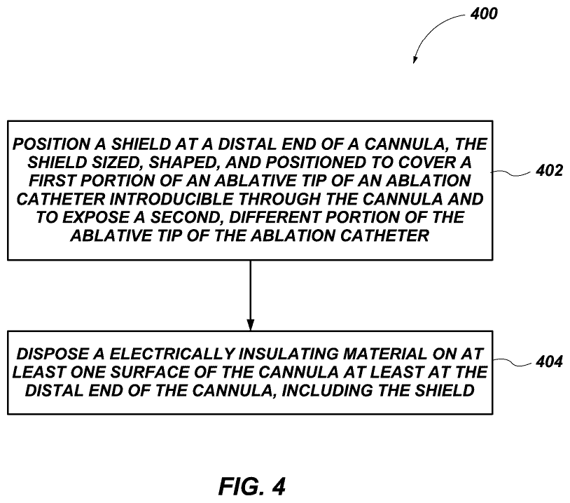

FIG. 4 is a flowchart showing a method 400 of making a protective working channel. The method 400 may involve, for example, positioning a shield at a distal end of a cannula, as shown at act 402. The shield may be sized, shaped, and positioned to cover a first portion of an ablative tip of an ablation probe introducible through the cannula and to expose a second, different portion of the ablative tip of the ablation probe, as also shown at act 402. The shield may be formed, for example, by removing material from a previously closed, distal end of the cannula, leaving the shield. As another example, the shield may be formed by selectively positioning material only partially around a circumference of the cannula, such as, for example, during an additive manufacturing process. An electrically insulating material may be disposed on at least one surface of the cannula at least at the distal end of the cannula, including the shield, as shown at act 404. The electrically insulating material may be disposed on one or more surfaces of the cannula utilizing, for example, a powder coat process, a dip and cure process, or by any other coating process known in the art or later developed.

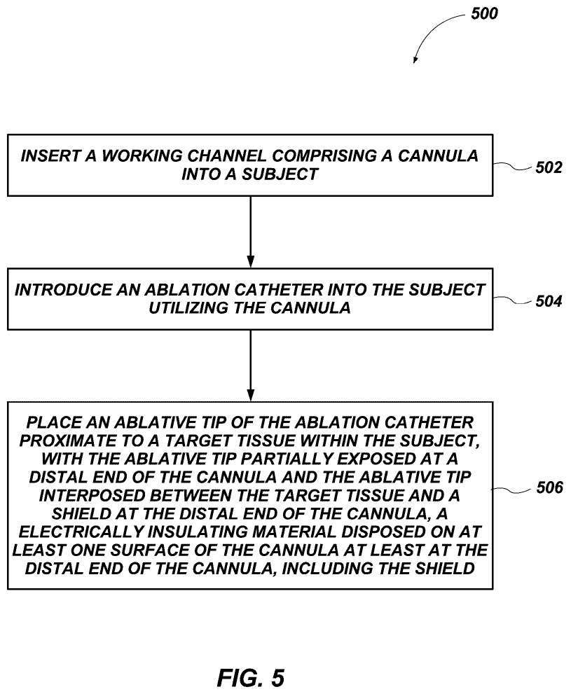

FIG. 5 is another flowchart showing a method 500 of using a protective working channel. The method 500 may involve inserting a protective working channel including a cannula into a subject, as indicated at act 502. An ablation probe may be introduced into the subject utilizing the cannula, as indicated at act 504. An ablative tip of the ablation probe may be placed proximate to a target tissue within the subject, as indicated at act 506. The ablative tip may be partially exposed at a distal end of the cannula and the ablative tip may be interposed between the target tissue and a shield at the distal end of the cannula, as further indicated at act 506. An electrically insulating material disposed on at least one surface of the cannula at least at the distal end of the cannula, including the shield, as also indicated at act 506.

When performing an ablation procedure, the shield may inhibit heat and other energy emittable from the ablation tip from ablating tissues other than a target tissue (e.g., radio frequency energy). For example, the shield, and the electrically insulating material of the shield, may result in a temperature in a tissue on a side of the shield opposite the ablative tip being about 100 degrees Fahrenheit (37.8 degrees Celsius) to about 250 degrees Fahrenheit (121.1 degrees Celsius) lower than a temperature in a target tissue. More specifically, the shield, and the electrically insulating material of the shield, may result in the temperature in the tissue on the side of the shield opposite the ablative tip being about 150 degrees Fahrenheit (65.6 degrees Celsius) to about 200 degrees Fahrenheit (93.3 degrees Celsius) (e.g., about 175 degrees Fahrenheit (79.4 degrees Celsius)) lower than the temperature in the target tissue.

Protective working channels in accordance with this disclosure may better facilitate precise ablation of target tissues and may reduce damage to heathy tissues proximate to the target tissues, by further focusing the directional targeting of energy emittable by an ablative tip of an ablation probe. Protective working channels in accordance with this disclosure may also enable more aggressive ablation of target tissue while mitigating risks to surrounding healthy tissues by inhibiting flow of heat and other energy emittable from the ablative tip toward the healthy tissue utilizing the shield of the protective working channel.

Additional, nonlimiting examples within the scope of this disclosure include:

Example 1: A protective working channel useful in an ablative procedure, the protective working channel comprising: a cannula comprising a shield at a distal end of the cannula, the shield sized, shaped, and positioned to cover a first portion of an ablative tip of an ablation probe introducible through the cannula and to expose a second, different portion of the ablative tip of the ablation probe; and an electrically insulating material disposed on at least one surface of the cannula at least at the distal end of the cannula, including the shield.

Example 2: The protective working channel of Example 1, wherein the cannula is sized and shaped to enable an introducer needle and the ablation probe to pass at least partially through the cannula.

Example 3: The protective working channel of Example 1 or Example 2, wherein the electrically insulating material is disposed on at least substantially all surfaces of the cannula.

Example 4: The protective working channel of any one of Examples 1 through 3, wherein the electrically insulating material is disposed on at least substantially all external surfaces of the cannula, at least substantially all internal surfaces of the cannula, or at least substantially all external and at least substantially all internal surfaces of the cannula.

Example 5: The protective working channel of any one of Examples 1 through 4, further comprising a handle at a proximal end of the cannula.

Example 6: The protective working channel of any one of Examples 1 through 5, wherein the cannula comprises a biocompatible material.

Example 7: The protective working channel of Example 6, wherein the cannula comprises stainless steel.

Example 8: The protective working channel of any one of Examples 1 through 7, wherein the cannula is rigid.

Example 9: The protective working channel of any one of Examples 1 through 8, wherein the cannula is flexible.

Example 10: The protective working channel of any one of Examples 1 through 9, wherein the electrically insulating material comprises porcelain.

Example 11: The protective working channel of any one of Examples 1 through 9, wherein the electrically insulating material comprises a polymer material.

Example 12: The protective working channel of Example 11, wherein the electrically insulating material comprises a polyester material.

Example 13: The protective working channel of any one of Examples 1 through 12, wherein the shield is sized, shaped, and positioned to cover about half of an exterior of the ablative tip or greater, and to expose a remainder of the ablative tip, when the ablative tip is located proximate to the shield and positioned for an ablation procedure.

Example 14: The protective working channel of any one of Examples 1 through 13, wherein an inner diameter of the cannula is sized to receive a 16 gauge or larger probe within the cannula.

Example 15: A kit for performing an ablation procedure, comprising: a protective working channel comprising: a cannula comprising a shield at a distal end of the cannula; and an electrically insulating material disposed on at least one surface of the cannula at least at the distal end of the cannula, including the shield; an introducer needle introducible through the cannula; and an ablation probe introducible through the cannula, through the introducer needle, or through the cannula and through the introducer needle; wherein the shield is sized, shaped, and positioned to cover a first portion of an ablative tip of the ablation probe and to expose a second, different portion of the ablative tip of the ablation probe when the ablation probe is at least partially inserted into the cannula, the ablative tip is located proximate to the distal end of the cannula, and the ablative tip is oriented for performing an ablation procedure.

Example 16: The kit of Example 15, wherein a lesion wire of the ablative tip is exposed at a first angular position around a circumference of the ablative tip, and wherein a second angular position of the ablative tip, offset from the first angular position by 180 degrees, lacks exposed lesion wire.

Example 17: A method of making a protective working channel, the method comprising: positioning a shield at a distal end of a cannula, the shield sized, shaped, and positioned to cover a first portion of an ablative tip of an ablation probe introducible through the cannula and to expose a second, different portion of the ablative tip of the ablation probe; and disposing an electrically insulating material on at least one surface of the cannula at least at the distal end of the cannula, including the shield.

Example 18: A method of using a protective working channel, comprising: inserting a protective working channel comprising a cannula into a subject; introducing an ablation probe into the subject utilizing the cannula; and placing an ablative tip of the ablation probe proximate to a target tissue within the subject, with the ablative tip partially exposed at a distal end of the cannula and the ablative tip interposed between the target tissue and a shield at the distal end of the cannula, an electrically insulating material disposed on at least one surface of the cannula at least at the distal end of the cannula, including the shield.

Example 19: The method of Example 18, further comprising ablating the target tissue utilizing the ablative tip.

Example 20: The method of Example 18 or Example 19, further comprising inhibiting ablation of tissue other than the target tissue utilizing the shield.

While certain illustrative embodiments have been described in connection with the figures, those of ordinary skill in the art will recognize and appreciate that the scope of this disclosure is not limited to those embodiments explicitly shown and described in this disclosure. Rather, many additions, deletions, and modifications to the embodiments described in this disclosure may be made to produce embodiments within the scope of this disclosure, such as those specifically claimed, including legal equivalents. In addition, features from one disclosed embodiment may be combined with features of another disclosed embodiment while still being within the scope of this disclosure.

What is claimed is:

1. A protective working channel useful in an ablative procedure, the protective working channel comprising:
a cannula comprising a shield at a distal end of the cannula, the shield sized, shaped, and positioned to cover a first portion of an ablative tip of an ablation probe introducible through the cannula and to expose a second, different portion of the ablative tip of the ablation probe; and
an electrically insulating material disposed on at least one surface of the cannula at least at the distal end of the cannula, including the shield.

2. The protective working channel of claim 1, wherein the cannula is sized and shaped to enable an introducer needle and the ablation probe to pass at least partially through the cannula.

3. The protective working channel of claim 1, wherein the electrically insulating material is disposed on at least substantially all surfaces of the cannula.

4. The protective working channel of claim 1, wherein the electrically insulating material is disposed on at least substantially all external surfaces of the cannula, at least substantially all internal surfaces of the cannula, or at least substantially all external and at least substantially all internal surfaces of the cannula.

5. The protective working channel of claim 1, further comprising a handle at a proximal end of the cannula.

6. The protective working channel of claim 1, wherein the cannula comprises a biocompatible material.

7. The protective working channel of claim 6, wherein the cannula comprises stainless steel.

8. The protective working channel of claim 1, wherein the cannula is rigid.

9. The protective working channel of claim 1, wherein the cannula is flexible.

10. The protective working channel of claim 1, wherein the electrically insulating material comprises porcelain.

11. The protective working channel of claim 1, wherein the electrically insulating material comprises a polymer material.

12. The protective working channel of claim 11, wherein the electrically insulating material comprises a polyester material.

13. The protective working channel of claim 1, wherein the shield is sized, shaped, and positioned to cover about half of an exterior of the ablative tip or greater, and to expose a remainder of the ablative tip, when the ablative tip is located proximate to the shield and positioned for an ablation procedure.

14. The protective working channel of claim 1, wherein an inner diameter of the cannula is sized to receive a 16 gauge or larger probe within the cannula.

15. A kit for performing an ablation procedure, comprising:
a protective working channel comprising:
a cannula comprising a shield at a distal end of the cannula; and
an electrically insulating material disposed on at least one surface of the cannula at least at the distal end of the cannula, including the shield;
an introducer needle introducible through the cannula; and
an ablation probe introducible through the cannula, through the introducer needle, or through the cannula and through the introducer needle;
wherein the shield is sized, shaped, and positioned to cover a first portion of an ablative tip of the ablation probe and to expose a second, different portion of the ablative tip of the ablation probe when the ablation probe is at least partially inserted into the cannula, the ablative tip is located proximate to the distal end of the cannula, and the ablative tip is oriented for performing an ablation procedure.

16. The kit of claim 15, wherein a lesion wire of the ablative tip is exposed at a first angular position around a circumference of the ablative tip, and wherein a second angular position of the ablative tip, offset from the first angular position by 180 degrees, lacks exposed lesion wire.

17. A method of making a protective working channel, the method comprising:
positioning a shield at a distal end of a cannula, the shield sized, shaped, and positioned to cover a first portion of an ablative tip of an ablation probe introducible through the cannula and to expose a second, different portion of the ablative tip of the ablation probe; and
disposing an electrically insulating material on at least one surface of the cannula at least at the distal end of the cannula, including the shield.

18. A method of using a protective working channel, comprising:
inserting a protective working channel comprising a cannula into a subject;
introducing an ablation probe into the subject utilizing the cannula; and
placing an ablative tip of the ablation probe proximate to a target tissue within the subject, with the ablative tip partially exposed at a distal end of the cannula and the ablative tip interposed between the target tissue and a shield at the distal end of the cannula, an electrically insulating material disposed on at least one surface of the cannula at least at the distal end of the cannula, including the shield.

19. The method of claim 18, further comprising ablating the target tissue utilizing the ablative tip.

20. The method of claim 18, further comprising inhibiting ablation of tissue other than the target tissue utilizing the shield.
